(19) United States
(12) Patent Application Publication
(10) Pub. No.: US 2022/0280090 A1
(43) Pub. Date: Sep. 8, 2022

(54) BODY CONDITION MEASUREMENT TAPE AND METHOD FOR PROVIDING BODY CONDITION MONITORING SERVICE BY USING SAME

(71) Applicant: NEWTON1665 Co., Ltd, Seoul (KR)

(72) Inventor: Mi Ra PARK, Seoul (KR)

(21) Appl. No.: 17/633,475

(22) PCT Filed: Nov. 17, 2019
(86) PCT No.: PCT/KR2019/015715

§ 371 Date: Feb. 7, 2022

(30) Foreign Application Priority Data
Aug. 5, 2019 (KR) 10-2019-0095241

(51) Int. Cl. A61B 5/22; A61B 5/11; A61B 5/00; A61B 5/08

(52) U.S. Cl. CPC A61B 5/228; A61B 5/1114; A61B 5/4818; A61B 5/682; A61B 5/4561; A61B 5/0002; A61B 5/0816

(57) ABSTRACT

The present disclosure relates to a method of providing a physical condition monitoring service using a physical condition measurement tape comprising a tape body selectively attached to a body and a physical condition measurement sensor provided on the tape body, a device, and a server communicating with the device. The method includes performing first data transmission by wirelessly transmitting physical condition data from the physical condition measurement tape to the device, performing second data transmission by transmitting the physical condition data from the device to the server, processing the physical condition data by analyzing and processing, in the server, the physical condition data into physical condition information, and performing physical condition information transmission by transmitting the physical condition information from the server to the device.

### BACKGROUND

The present disclosure relates to a physical condition measurement tape and a method of providing a physical condition monitoring service using the same.

Anatomically, the muscles of the human body have to form a closed vector system such that the sum of tensile force, compressive force, and torsional stress becomes zero at any moment when lying, sitting, standing, walking, or running to achieve stability.

Conversely, when muscles form an open vector system, a tensile force, compressive force, and torsional stress are concentrated on a specific part of the body, resulting in chronic pain in the part of the body, or various other diseases, such as malocclusion of teeth, or snoring, sleep apnea, rhinitis, facial asymmetry, forward neck or turtle neck syndrome, spinal discs, or strabismus, due to body imbalance.

In recent years, the number of people having the above-described body imbalance disease has been increasing day by day, and in order to solve the problem, a “Lip Seal Tape (KR Application No. 10-2017-0154558)” has been filed.

In the related art, the main feature is to increase the body stability and promote nasal breathing is promoted by preventing the upper lip and the lower lip from opening so that the lip periphery is maintained in the closed vector system.

However, the related art may not have functions other than the function of maintaining the vicinity of the lips as a closed vector, and thus it has a clear limitation in terms of its use.

In addition, even if the lip seal tape in the related art is used, it takes a considerable amount of time (about six months to three years) to completely correct the body imbalance, and as a result, it is difficult for the user to immediately recognize the improvement effect with the use, which may be a limitation that the user is difficult to wear it continuously with patience.

Therefore, a more advanced way than the related art is needed to measure the state of body imbalance, correct the imbalance, and at the same time, monitor the effect immediately, and is also needed to more diversify its use so as to be used on other parts of the body other than the lips.

The skeleton is radiographed only in the lying position without objective data on what magnitude and direction of force a subject receives and what kind of deformation the force causes in a specific posture, and the skeletal status in pain is not checked, which may make it difficult to determine the exact cause of musculoskeletal disorders.

The skeleton is radiographed only in the lying position without objective data on what magnitude and direction of force a subject receives and what kind of shape change the force causes when the subject receives a specific treatment and the skeletal statuses before and after radiographing are compared with each other, which may make it difficult to perform accurate non-surgical conservative treatment on musculoskeletal disorders.

### SUMMARY

The present disclosure has been devised to solve the above-mentioned problems in the related art, and is intended to monitor an imbalance state of the body, and more particularly, intended to measure the muscle strength distribution state of the body by using a tape having a simple structure and to provide data therefor.

In addition, the present disclosure is intended to allow a user to check a magnitude and direction of a force acting on the shape change of the entire human body structure by identifying, when a specific muscle generates a force of a specific direction and magnitude, an interrelationship with other muscles.

In addition, the present disclosure is intended to provide a user with a risk of musculoskeletal disease by identifying a site where pressure is concentrated in a specific posture.

In addition, the present disclosure is intended to correct the imbalance of the body, and at the same time, to allow the user to check the process of correcting the imbalance.

In addition, the present disclosure is intended to monitor a sleep state.

In accordance with an exemplary embodiment of the present disclosure, there is provided a method of providing a physical condition monitoring service using a physical condition measurement tape including a tape body selectively attached to a body and a physical condition measurement sensor provided on the tape body, a device, and a server communicating with the device, and the method includes performing first data transmission by wirelessly transmitting physical condition data from the physical condition measurement tape to the device, performing second data transmission by transmitting the physical condition data from the device to the server, performing physical condition data processing by analyzing and processing, in the server, the physical condition data into physical condition information, and performing physical condition information transmission by transmitting the physical condition information from the server to the device.

The physical condition measurement sensor may include a muscle sensor configured to measure a tensile force formed on a body part of a user to which the tape body is attached, and the physical condition information may include an average tensile force during a measurement time.

The muscle sensor may be provided on the tape body so that a longitudinal direction thereof intersects upper and lower lips of the user sequentially to detect mouth opening of the user, and the physical condition information may include a time or number of times of mouth opening.

The physical condition measurement sensor may include a muscle sensor configured to measure a torsional stress formed on a body part of a user to which the tape body is attached, and the physical condition information may include an average torsional stress during a measurement time.

The physical condition measurement sensor may include a breathing sensor configured to measure a breathing volume and breathing cycle during sleep of a user, and the physical condition information may include a time or number of times of sleep apnea.

The physical condition measurement sensor may include a microphone configured to measure snoring that occurs during sleep of a user, and the physical condition information may include at least one of a time, a size, and the number of times of snoring.

The server may further include a storage part configured to store the physical condition data and the physical condition information that are accumulated, and the accumulated physical condition information may be provided through the device.

The server may further include a statistics management part configured to process the accumulated physical condition data, and the statistics management part may process the physical condition data into the physical condition information through extraction, calculation, and analysis of valid data.

The server may further include a statistics management part configured to graph the physical condition data that is accumulated, and the graph generated by the statistics management part may be provided through the device.

The method may further include: performing photograph transmission by transmitting a skeleton photograph of a user from the device to the server; performing skeletal data processing by analyzing the body skeleton photograph and deriving skeletal information numerically indicating a state of skeletal balance, in the server; and performing skeleton information transmission by transmitting skeleton information from the server to the device.

In accordance with another exemplary embodiment of the present disclosure, a physical condition measurement tape includes: a tape body selectively attached to a skin; a physical condition measurement sensor provided on the tape body; and a communication part provided on the tape body and configured to wirelessly transmit a value measured by the physical condition measurement sensor to an external device.

### BRIEF DESCRIPTION OF THE DRAWINGS

FIG. 1 is a schematic diagram of a physical condition measurement tape in accordance with an exemplary embodiment of the present disclosure;

FIG. 2 is a diagram illustrating an example in which the physical condition measurement tape is attached to a face of a user;

FIG. 3 is a diagram illustrating an example in which the physical condition measurement tape is attached to a neck of the user;

FIG. 4 is a diagram illustrating an example in which the physical condition measurement tape is attached to a shoulder of the user;

FIG. 5 is a diagram illustrating an example in which the physical condition measurement tape is attached to a waist of the user;

FIG. 6 is a diagram illustrating an example in which the physical condition measurement tape is attached to a leg of the user;

FIG. 7 is a schematic diagram of a system for implementing a method of providing a physical condition monitoring service using the physical condition measurement tape;

FIG. 8 is a block diagram illustrating a detailed configuration of the system for implementing the method of providing the physical condition monitoring service;

FIG. 9 illustrates a sleep condition measurement procedure and a body muscle condition measurement procedure in the method of providing the physical condition monitoring service;

FIG. 10 illustrates a skeletal change analysis procedure in the method of providing the physical condition monitoring service;

FIG. 11 illustrates a detailed procedure of a muscle condition data processing operation;

FIG. 12 is a diagram illustrating a main page provided to a user equipment by the method of providing the physical condition monitoring service in accordance with an exemplary embodiment of the present disclosure;

FIGS. 13 to 14 are diagrams illustrating pages for monitoring a sleep state, which are provided to a user by the method of providing the physical condition monitoring service in accordance with an exemplary embodiment of the present disclosure;

FIGS. 15 to 19 are diagrams illustrating pages for monitoring a muscle state, which are provided to a user by the method of providing the physical condition monitoring service in accordance with an exemplary embodiment of the present disclosure;

FIGS. 20 to 23 are diagrams illustrating pages for monitoring a skeletal change state, which are provided to a user by the method of providing the physical condition monitoring service in accordance with an exemplary embodiment of the present disclosure; and

FIGS. 24 to 27 are diagrams illustrating pages provided to an administrator by the method of providing the physical condition monitoring service in accordance with an exemplary embodiment of the present disclosure.

### DETAILED DESCRIPTION OF EMBODIMENTS

Hereinafter, various exemplary embodiments of the present disclosure will be described with reference to the drawings.

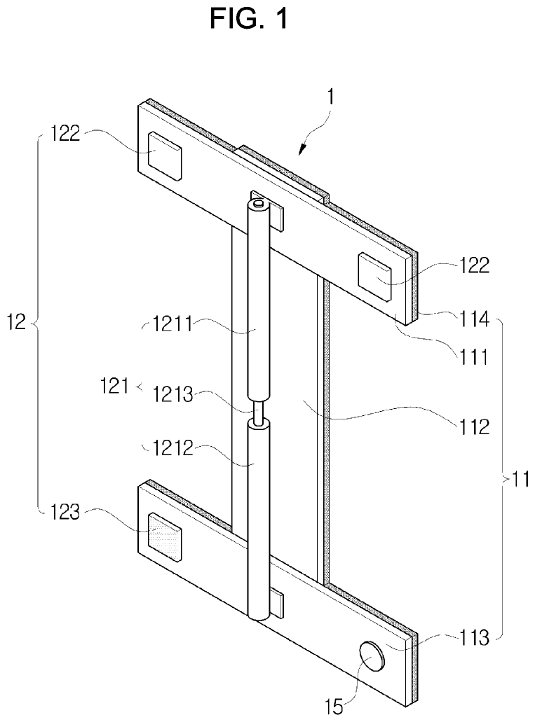

First, a physical condition measurement tape 1 in accordance with an exemplary embodiment of the present disclosure will be described with reference to FIG. 1.

The physical condition measurement tape 1 is used by simply attaching to the skin of a user, and may be provided to measure the tensile force and torsional stress acting on the attached body part, and the physical condition such as the breathing cycle and snoring.

Specifically, the physical condition measurement tape 1 may include a tape body 11 selectively attached to the body of the user, and the tape body 11 may include a first body 111, a second body 112 extending downward from the center of the first body 111, and a third body 113 extending from the lower end of the second body 112 to both sides.

That is, the tape body 11 may be provided in a shape in which ‘H’ is rotated by 90 degrees, and the rear surface of each body may be provided with a reusable adhesive part 114 that is attached and fixed to the skin of the user and confirmed for skin safety.

The first to third bodies may be integrally formed or may be individually formed and coupled to each other during a manufacturing process.

The physical condition measurement tape 1 may further include a physical condition measurement sensor 12 provided on the tape body 11 to measure the physical condition, and a communication part 15 provided on the tape body 11 to wirelessly transmit, to an external device, a value sensed by the physical condition measurement sensor 12.

In more detail, the physical condition measurement sensor 12 may include a muscle sensor 121 that measures the tensile force and torsional stress of the body part of the user to which the tape body 11 is attached.

That is, the muscle sensor 121 is easily attached to the skin of the user to measure the tensile force and torsional stress formed in the muscle of the attachment part, and the measured result may be transmitted to an external device through the communication part 15.

To this end, the muscle sensor 121 may further include a first fixing part 1211 fixed to the first body 111, a second fixing part 1212 fixed to the second body 112, and a connection part 1213 connecting the first fixing part 1211 and the second fixing part 1212.

When the muscle sensor 121 indicates that the first fixing part 1211 and the second fixing part 1212 move away from the center of the connection part 1213, it may be determined that the muscle is tensioned, and when the muscle sensor 121 indicates the opposite, that is, they are approaching the center, it may be determined that the muscle is compressed.

In addition, when the first fixing part 1211 and the second fixing part 1212 rotate in opposite directions about the longitudinal axis of the connection part 1213, it may be determined that torsional stress is acting on the muscle.

Meanwhile, the physical condition measurement sensor 12 may further include breathing sensors 122 provided on both sides of the first body 111 to measure a breathing cycle during sleep of the user.

Specifically, a humidity sensor or a pressure sensor may be provided as the breathing sensor 122; when the humidity sensor is provided, an exhalation and inhalation cycle may be determined by using the principle that the humidity temporarily increases due to the moisture contained in the exhalation, and when the pressure sensor is provided, the exhalation and inhalation cycle may be determined by using the principle that the pressure is temporarily increased by the exhalation.

The physical condition measurement sensor 12 may further include a microphone 123 provided on the third body 113 to measure snoring that occurs during sleep of the user, and when a sound having a frequency band of a predetermined range is transmitted to the microphone 123, the physical condition may be recognized as snoring.

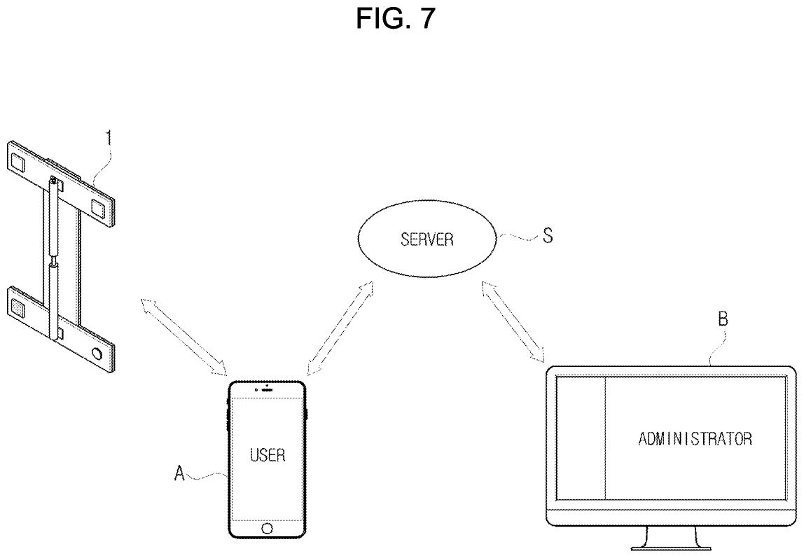

A control part is provided in the tape body 11 so that data measured by the physical condition measurement sensor 12 on the physical condition measurement tape 1 may be processed into the above-mentioned physical condition information; however, in the present exemplary embodiment, it is desirable to transmit the data to the outside through the communication part and process it in external equipment in order to construct a physical condition monitoring system that a user may check through an external device and efficiently utilize resources. This will be described in detail later with reference to FIGS. 7 to 9.

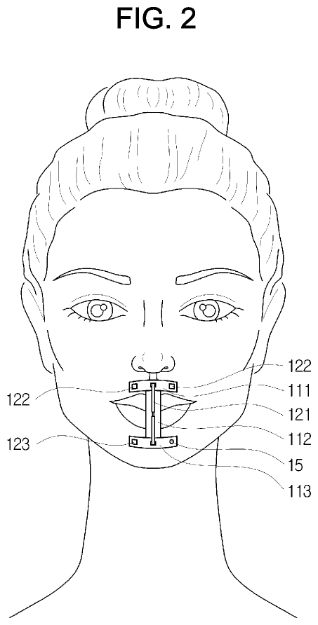

Next, FIG. 2 illustrates an example in which the physical condition measurement tape 1 is attached over upper and lower lips of the user.

The tape body 11 is vertically attached along the running line of the muscle of the closed mouth to close the superficial anterior line (upper and lower lips) and the deep anterior line (tongue and roof of the mouth), so that the superficial anterior line and the deep anterior line may be closed curves, and the vector sum of the superficial anterior line and the deep anterior line may be 0, accordingly.

That is, the tape body 11 in accordance with the present disclosure may form a closed vector system of whole body muscles by adding a closed vector in the superficial anterior line and the deep anterior line to help the vector displacing the position of the tongue in the anterior superior direction, and thus may increase body stability by balancing the forces and change the muscle vector forming a neutral zone in a physiological direction of each user over time.

In addition, since the muscle sensor 121 is provided on the tape body 11 so that its longitudinal direction intersects the upper and lower lips of the user sequentially, mouth opening of the user may be sensed by measuring the tensile force.

In this case, the breathing sensor 122 may be located below the nostril of the user, and the snoring sound of the user may be measured by the microphone 123.

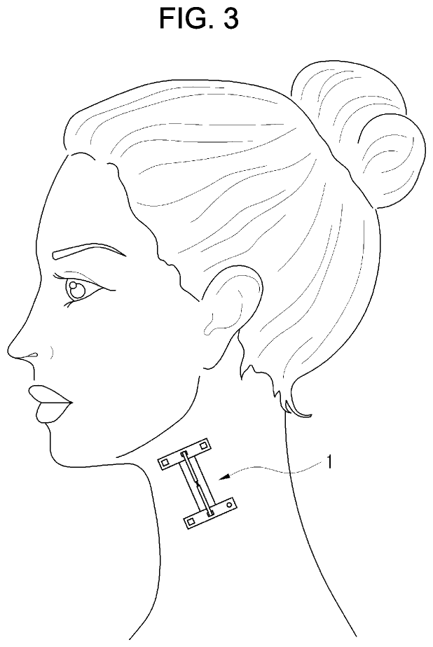

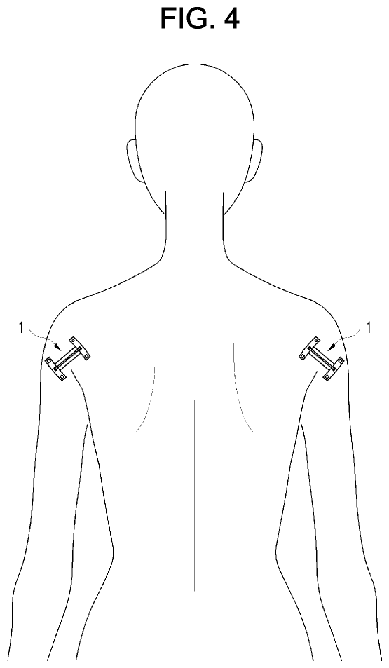

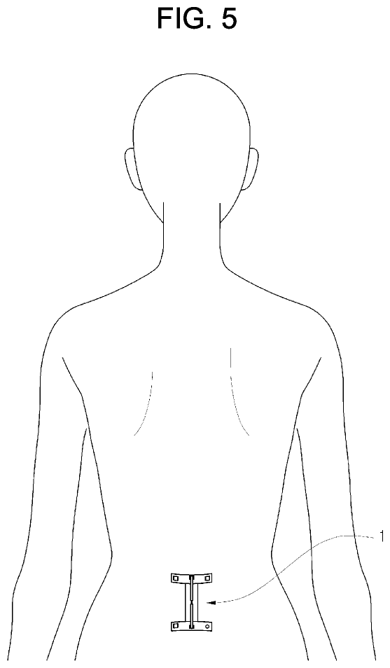

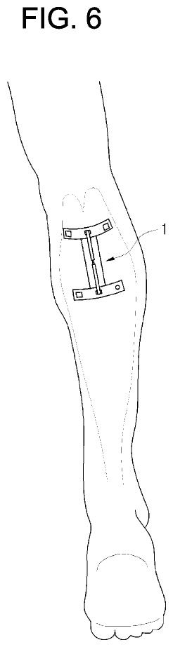

Meanwhile, FIG. 3 is an example in which the physical condition measurement tape 1 is attached to the neck part of the user, FIG. 4 is an example in which the physical condition measurement tape 1 is attached around the scapula from the shoulder of the user, and FIG. 5 is an example in which the physical condition measurement tape 1 is attached to the waist, and FIG. 6 is an example in which the physical condition measurement tape 1 is attached to the calf.

That is, as can be seen in FIGS. 3 to 6, the physical condition measurement tape 1 may be attached to any part of the body of the user as needed, and the tensile force and torsional stress formed in the muscle near the attached part are measured and data on the measurement may be transmitted to the outside.

Hereinafter, a method of providing a physical condition monitoring service will be described in which the above-mentioned physical condition data is transmitted to the outside, the physical condition data transmitted to the outside is processed into physical condition information, and the physical condition information is provided through a user device.

FIG. 7 is a schematic diagram of a physical condition monitoring system for implementing a method of providing a physical condition monitoring service.

The physical condition monitoring system in accordance with an exemplary embodiment of the present disclosure may include a server S capable of being accessed through wire or wireless communication and storing information, the physical condition measurement tape 1, a user device A capable of accessing the sever S through wired or wireless communication and receiving physical condition information from the physical condition measurement tape 1, and an administrator device B capable of accessing the server S through wired or wireless communication.

In this case, the user may be an individual, medical institution, or the like, and an administrator may be a medical institution, government agency, physical condition monitoring service provider, and the like. In addition, a plurality of the user devices A and a plurality of the administrator devices B may be connected to the server S as needed.

Here, the user device A and the administrator device B may refer to any unit including equipment capable of transmission through a wired or wireless Internet network.

That is, the devices correspond to pieces of equipment for access through the Internet, and broadly, may include an input device, such as keyboard and mouse, for inputting various data, an output device such as a monitor and printer, a transceiver such as a modem or LAN, a processing device such as a microprocessor or graphic card, and a storage equipped with various programs for operating or controlling them.

Examples of such devices may include a personal communication system (PCS) device, a global system for mobile communications (GSM) device, a personal digital cellular (PDC) device, a personal handy-phone system (PHS) device, a personal digital assistant (PDA) device, a smart phone, telematics, a wireless data communication device and/or a portable Internet device, a personal computer, a laptop computer, and the like.

The physical condition data measured by the physical condition measurement tape 1 may be transmitted to the user device A through the communication part 15, and the transmitted physical condition data may be transmitted and processed to the server S through a wired/wireless communication network.

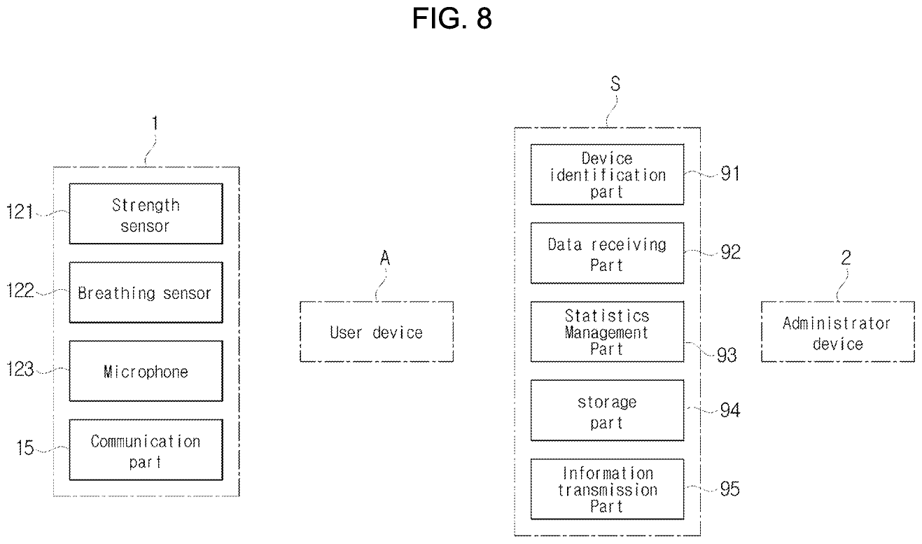

Individual configurations of the server S for accomplishing the above will be described in detail with reference to FIG. 8 below.

The server S may include a device identification part 91 that identifies the user of the user device A which is communicating, a data receiving part 92 that receives physical condition data from the user device A, a statistics management part 93 that processes the received physical condition data and derives physical condition information, a storage part 94 that accumulates and stores the physical condition data and physical condition information about the user, and an information transmission part 95 that transmits the physical condition information to the user device A.

The server S may be formed with a dedicated system and a wired/wireless communication network for implementing the method of providing the physical condition monitoring service, and may as well be implemented through a virtual file-based web cloud service.

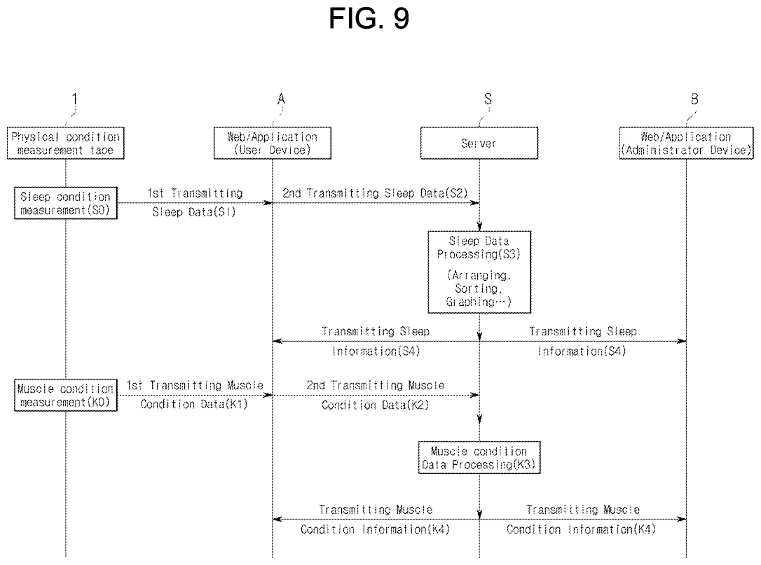

Hereinafter, the method of providing the physical condition monitoring service in accordance with an exemplary embodiment of the present disclosure will be described in detail with reference to FIG. 9.

The method of providing the physical condition monitoring service in accordance with an exemplary embodiment of the present disclosure may include a physical condition measurement step S0, K0 in which the physical condition is measured in the physical condition measurement tape 1, a first data transmission step S1, K1 in which physical condition data is wirelessly transmitted from the physical condition measurement tape 1 to the user device A, a second data transmission step S2, K2 in which the physical condition data is transmitted from the user device A to the server S, a physical condition data processing step S3, K3 in which the physical condition data is analyzed and processed into physical condition information in the statistics management part 93 of the server S, and a physical condition information transmission step S4, K4 in which the physical condition information is transmitted from the server S to the user device A and the administrator device B.

In more detail, a physical condition monitoring procedure using the physical condition measuring tape 1 may be divided into a sleep condition monitoring procedure and a muscle condition monitoring procedure.

First, the sleep condition monitoring procedure proceeds when the physical condition measurement tape 1 is attached to the lips of the user as shown in FIG. 2, and may include steps S0 to S4 shown in FIG. 9.

Specifically, the procedure may include a sleep condition step S0, a first data transmission step S1 in which sleep condition data is wirelessly transmitted from the physical condition measurement tape 1 to the user device A, a second data transmission step S2 in which the sleep condition data is transmitted from the user device A to the server S, a sleep condition data processing step S3 in which the sleep condition data is analyzed and processed into sleep condition information in the statistics management part 93 of the server S, and a sleep condition information transmission step S4 in which the sleep condition information is transmitted from the server S to the user device A and the administrator device B.

The sleep condition information may include the time/the number of times of mouth opening measured by the muscle sensor 121, the time/number of times of sleep apnea measured by the breathing sensor 122, and the time/magnitude/number of times of snoring measured by the microphone 123.

In this case, data processing may be a process of sorting data by classifying sleep condition data for each force type/date, extracting effective data by removing noise (excessive tensile force, compressive force or torsional stress temporarily generated by writhing, external noise, or the like) from the classified sleep condition data, calculating an average value or change value of the sleep condition data from which noise has been removed, and graphing the sleep condition based on the calculated value.

Through the above-described sleep condition monitoring procedure, it is possible for the user to easily receive the analyzed sleep condition information by attaching only the physical condition measurement tape 1 without a complicated device, and also to monitor the progress of gradually improving the sleep condition due to the closed vector system formed by the physical condition measurement tape 1.

Next, the muscle condition monitoring procedure may include steps K0 to K4 shown in FIG. 9.

Specifically, the procedure may include a muscle condition step K0, a first data transmission step K1 in which muscle condition data is wirelessly transmitted from the physical condition measurement tape 1 to the user device A, a second data transmission step K2 in which the muscle condition data is transmitted from the user device A to the server S, a muscle condition data processing step K3 in which the muscle condition data is analyzed and processed into muscle condition information in the statistics management part 93 of the server S, and a muscle condition information transmission step K4 in which the muscle condition information is transmitted from the server S to the user device A and the administrator device B.

The muscle condition information may include tensile force, compressive force, torsional stress, or the like of the muscle part measured by the muscle sensor 121.

Likewise, through the above-described muscle condition monitoring procedure, it is possible for the user to easily receive the analyzed muscle strength distribution information without a complicated device, and to perform correction for preventing overall skeletal deformation when a muscular strength imbalance is identified in the process.

Furthermore, in the muscle condition data processing step K3, the force applied to other muscles when the position of a specific part is changed depending on a specific posture, such as a change value of contraction or relaxation of other muscles by the projecting of the head, or a change value of contraction or relaxation of other muscles when lip sealing is not performed, may be calculated.

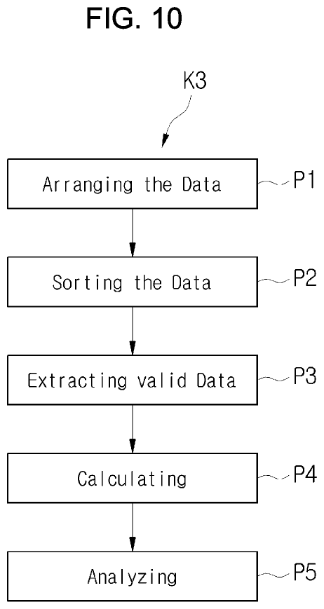

To this end, the muscle condition data processing step K3 may proceed according to a flowchart shown in FIG. 10.

Specifically, the muscle condition data processing step K3 may include a data arranging step P1 and a data sorting step P2 of classifying the received muscle condition data for each force type/muscle part/date, a valid data extraction step P3 of removing noise (excessive tensile force, compressive force or torsional stress temporarily generated by writhing, or the like) from the classified muscle condition data, a calculation step P4 of calculating an average value or change value for each force type (tensile force, compressive force, torsional stress) of the muscle condition data from which noise has been removed, and an analysis step P5 of calculating the degree of balance (or symmetry) in the left/right/up/down/axial direction of the body based on the calculated value and graphing the change.

Accordingly, based on the calculated information, the user may recognize that the user needs to improve his/her posture when an unstable posture is maintained, and improve the posture; conversely, when a good posture is maintained, the user may recognize that a stable posture is maintained.

Meanwhile, since the process of correcting the skeleton takes a rather long time as described in Background section, it is necessary to immediately quantify a state of skeletal balance and monitor it so that the user recognizes the correction effect and continuously performs the correction.

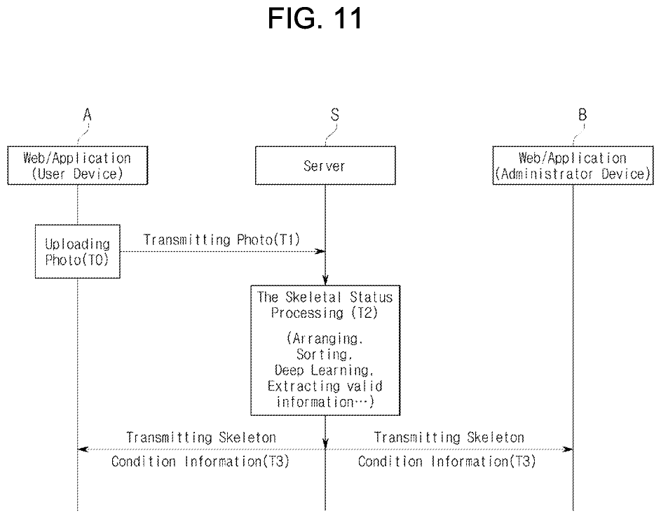

The skeletal status monitoring procedure provided to the user by analyzing changes in the skeleton, which is constructed according to this need, will be described below with reference to FIG. 11.

The skeletal status monitoring procedure is one type of the physical condition monitoring procedure, and may include a photograph upload step T0 in which a body skeleton photograph is uploaded to the upload user device A, a photograph transmission step T1 in which the uploaded body skeleton photograph is transmitted from the user device A to the server S, a skeletal data processing step T2 in which the body skeleton photograph is analyzed in the statistics management part 93 of the server S and skeleton condition information is derived in which the state of skeletal balance is expressed in terms of numbers, and a skeleton condition information transmission step T3 in which the skeleton condition information is transmitted from the server S to the user device A.

The skeleton condition information may include information on the degree of left-right balance, the degree of facial protrusion, the degree of uniform arrangement of teeth, the degree of body balance formation with respect to the inclination of the central axis penetrating the body in the longitudinal direction, and the like.

Accordingly, the user may receive skeletal information and obtain an excellent effect of grasping the trend of changes due to correction, simply by taking photographs of his/her craniofacial skeleton, teeth arrangement, waist, legs, entire body, or the like, and uploading them to a personal device.

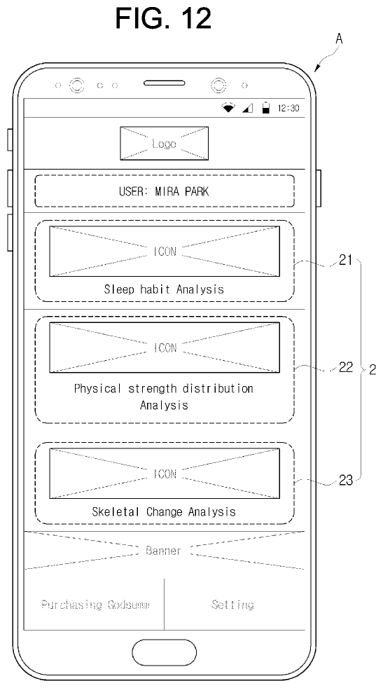

FIGS. 12 to 23 illustrate examples in which the above-described method of providing the physical condition monitoring service is implemented through a web/application of the user device A.

First, referring to FIG. 12, a main page 2 listing provided services may be provided on a display of the user device A, and on the main page 2, a sleep condition selection area 21 for monitoring sleep condition information, a muscle condition selection area 22 for monitoring muscle condition information, and a skeletal status selection area 23 for monitoring skeleton condition information are arranged in a partitioned manner.

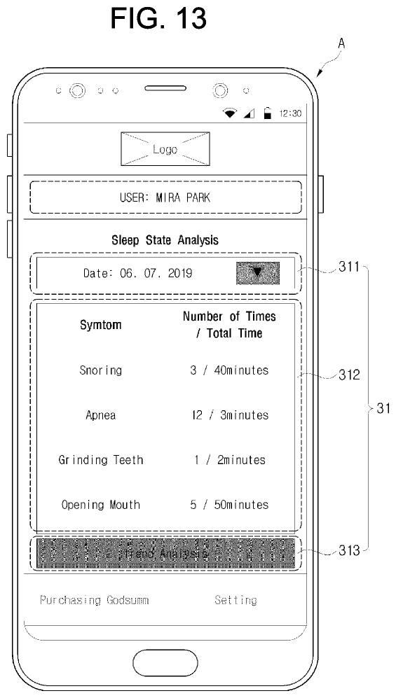

When the sleep condition selection area 21 is selected on the main page 2, a count page 31 illustrated in FIG. 13 may be provided on the display of the user device A.

In the count page 31, a date selection area 311 for selecting a date, a count area 312 for showing the sleep condition information corresponding to the selected date (time and number of times of snoring/sleep apnea/teeth grinding/mouth opening), and a trend analysis area 313 for turning to a trend analysis page 32 may be arranged in a partitioned manner.

Here, in the case of teeth grinding, when the tensile force and torsional stress of a predetermined magnitude or more are measured at predetermined intervals by the muscle sensor 121, the server S may determine teeth grinding.

Accordingly, when the user selects a date that the user wants to inquire, the sleep condition information for the day may be downloaded from the server S to the user device A so that the user inquires it.

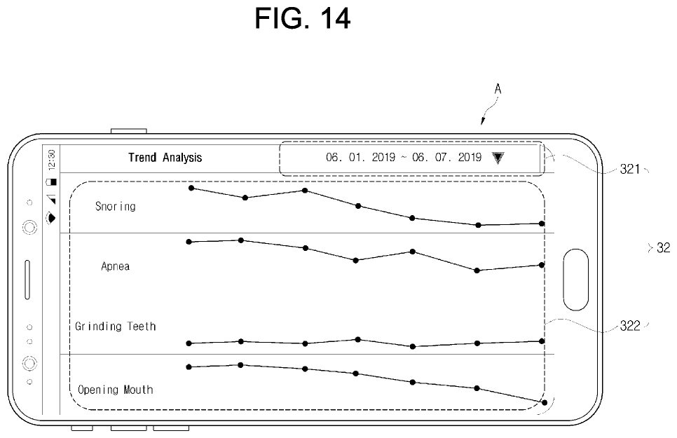

Next, FIG. 14 illustrates a graphed trend analysis page 32 in which the sleep state history of a specified period may be inquired, which is turned when the trend analysis area 313 is selected.

In the trend analysis page 32, a period area 321 for designating a period and a graphing area 322 for graphing the sleep condition information during the designated period may be arranged in a partitioned manner.

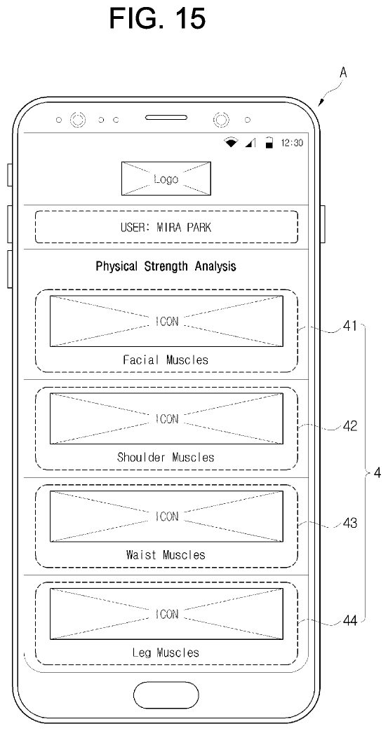

Next, when the muscle condition selection area 22 is selected on the main page 2, a muscle selection page 35 illustrated in FIG. 15 may be provided on the display of the user device A.

The muscle selection page 31 is for selecting a muscle part for which muscle condition information is to be inquired, and in the muscle selection page 4, a facial muscle selection area 41 for turning to a facial muscle analysis page 51 by selection, a shoulder muscle selection area 42 for turning to a shoulder muscle analysis page 52, a back muscle selection area 43 for turning to a waist muscle analysis page 53, and a leg muscle selection area 44 for turning to a leg muscle analysis page 54 may be arranged in a partitioned manner.

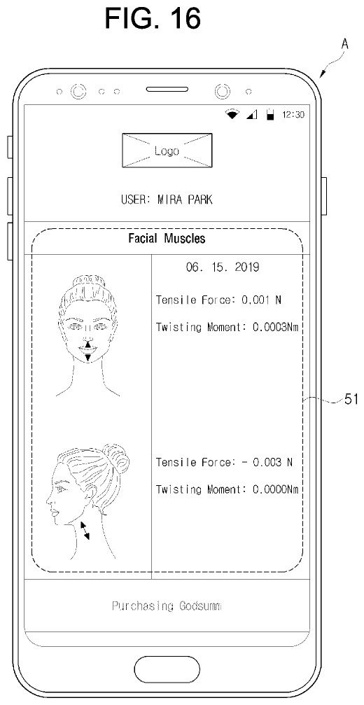

FIG. 16 illustrates the facial muscle analysis page 51, through which information on the average muscle tension and average muscle torsional stress for the facial part and the neck part to which the physical condition measurement tape 1 is attached may be provided.

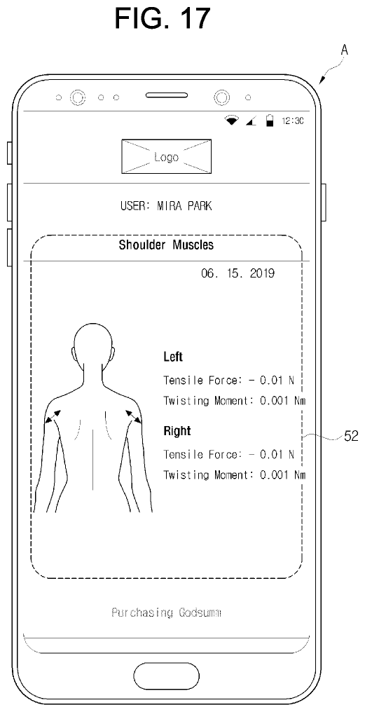

Similarly, FIG. 17 illustrates the shoulder muscle analysis page 52, through which information on the average muscle tension and average muscle torsional stress for the shoulder part to which the physical condition measurement tape 1 is attached may be provided.

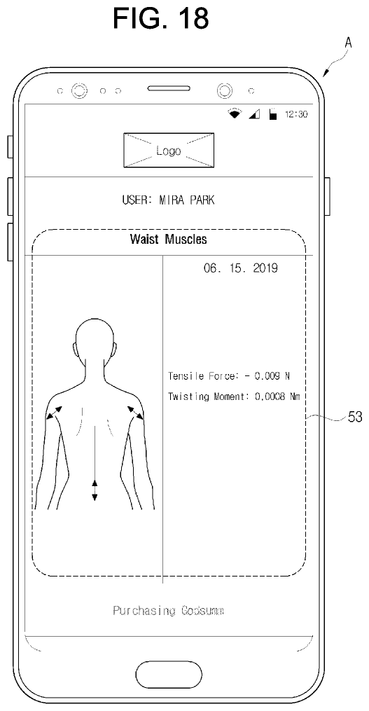

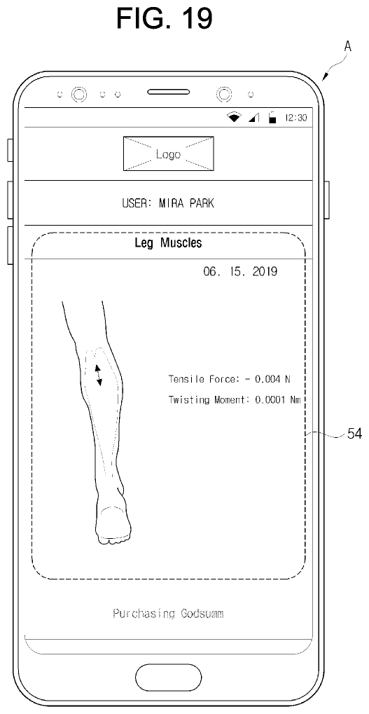

FIG. 18 illustrates the waist muscle analysis page 53, through which information on the average muscle tension and average muscle torsional stress for the waist part to which the physical condition measurement tape 1 is attached may be provided, and FIG. 19 illustrates the leg muscle analysis page 54, through which information on the average muscle tension and average muscle torsional stress for the leg part to which the physical condition measurement tape 1 is attached may be provided.

In the muscle analysis pages, information such as calculated force vectors and moments according to muscle conditions and skeletal positions may be provided cumulatively, and as a result, it is possible for the user to check the progress of correction as well as diagnosis of the current muscle condition at a glance.

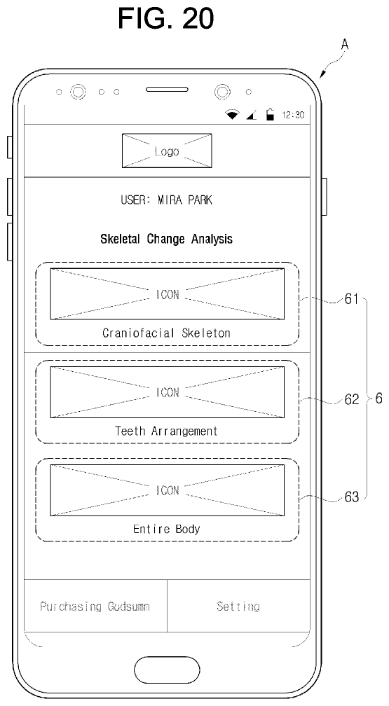

Next, when the skeletal status selection area 23 is selected on the main page 2, a skeleton selection page 6 illustrated in FIG. 20 may be provided on the display of the user device A.

The skeleton selection page 6 is for selecting a skeletal part for which skeleton condition information is to be inquired, and in the skeleton selection page 6, a craniofacial skeleton selection area 61 for switching to the craniofacial skeleton analysis page 71 by selection, a teeth arrangement selection area 62 for switching to a teeth arrangement analysis page 72, and an entire skeleton selection area 63 for switching to an entire skeleton analysis page 73 may be arranged in a partitioned manner.

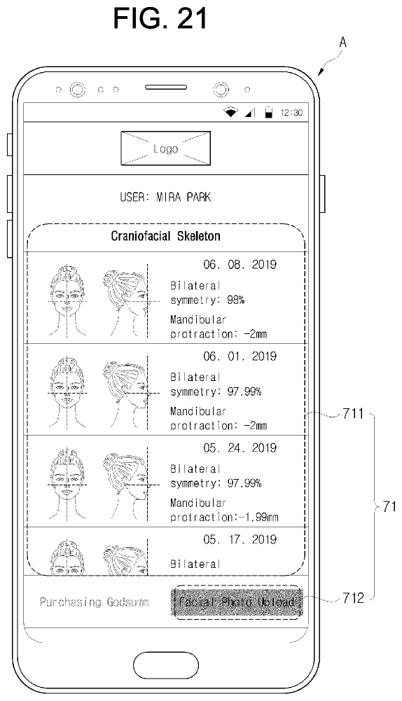

FIG. 21 illustrates the craniofacial skeleton analysis page 71, through which the craniofacial skeleton analysis area 711 provided with the left and right balance and protrusion state of the face in terms of numbers, and a facial photograph upload area 712 for uploading the face photograph may be arranged in a partitioned manner.

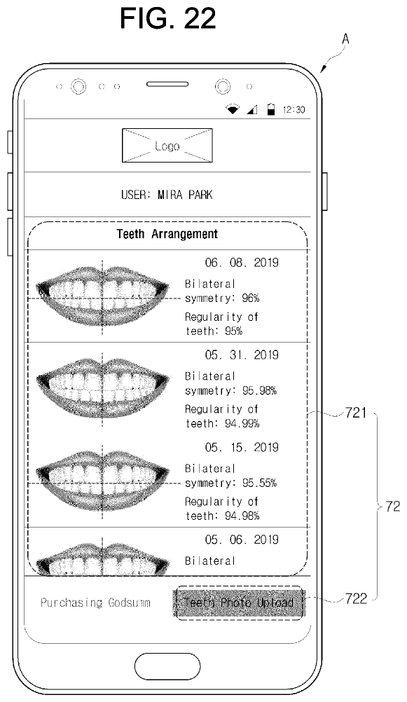

FIG. 22 illustrates the teeth arrangement analysis page 72, through which the teeth arrangement analysis area 721 provided with the left and right balance and evenness of the teeth in terms of numbers, and a teeth photograph upload area 722 for uploading the teeth arrangement photograph may be arranged in a partitioned manner.

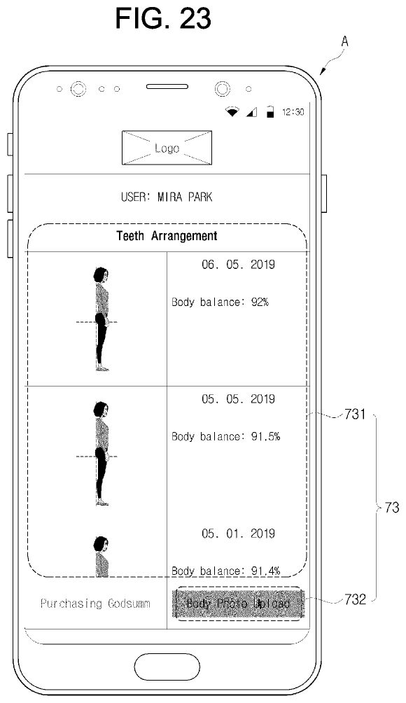

FIG. 23 illustrates the entire skeleton analysis page 73, through which the entire body skeleton area 731 provided with the body balance with respect to the inclination of the central axis penetrating the body in the longitudinal direction, in terms of numbers, and an entire body photograph upload area 732 for uploading the entire body photograph may be arranged in a partitioned manner.

When the user uploads, to the user device A, a photograph taken with the center or one end of the body part aligned with a guide line (dotted line), the photograph may be analyzed and expressed in terms of numbers in the skeletal data processing step T2, and then the skeleton condition information may be provided through the skeleton analysis pages 71, 72, and 73.

This configuration has an excellent effect of allowing the user to check the current skeletal balance state, as well as to simply inquire the process of skeletal change according to correction.

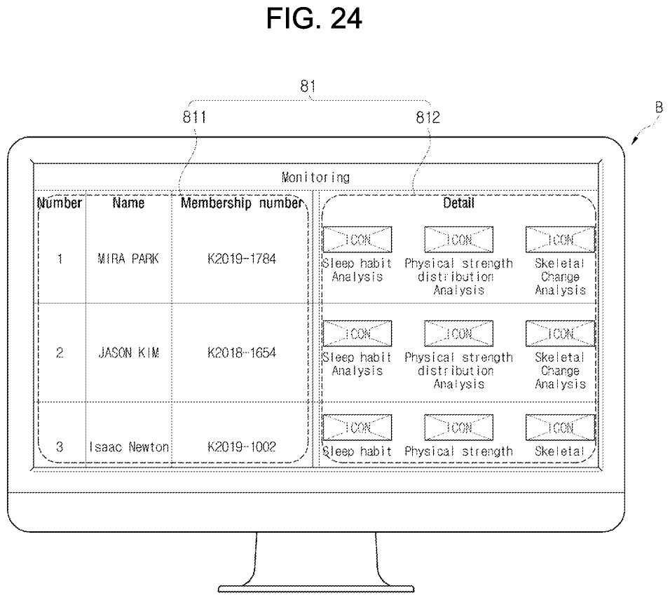

Next, FIGS. 24 to 27 illustrate an example in which the physical condition information is provided through the administrator device B, where it is characterized in that information provided from the user device A is provided and histories of several users may be selectively inquired.

First, referring to FIG. 24, all users may be inquired and a user management page 81 with a list of users may be viewed.

In the user management page 81, a user list area 811 in which user information is listed up and a physical condition inquiry selection area 812 for inquiring the physical condition of each user may be arranged in a partitioned manner.

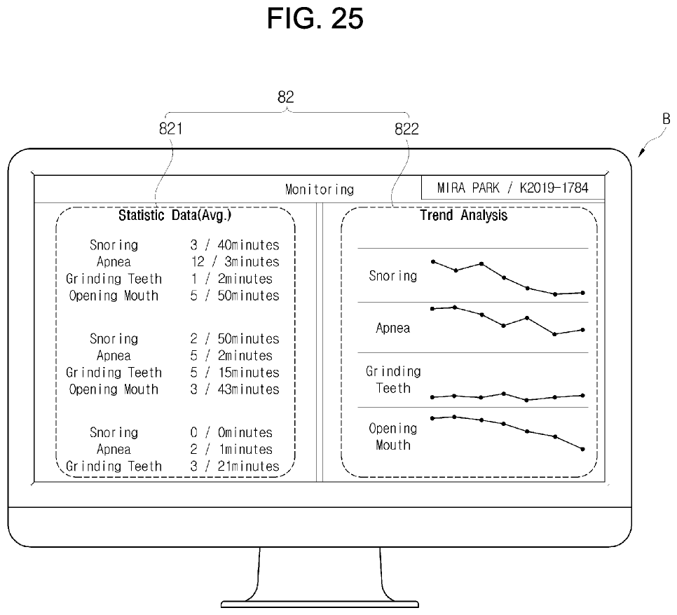

In FIG. 25, sleep condition information about the selected user may be provided to the user sleep condition management page 82 which is to be turned to when an area regarding the sleep state (Sleep Habit Analysis) is selected in the physical condition inquiry selection area 812.

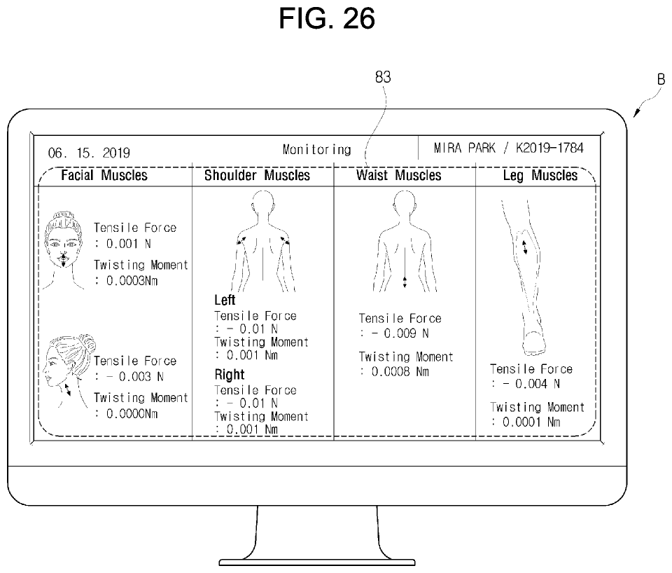

Further, in FIG. 26, muscle condition information about the selected user may be provided to the user muscle condition management page 83 which is to be turned to when an area regarding the muscle condition (Physical Strength Distribution) is selected in the physical condition inquiry selection area 812.

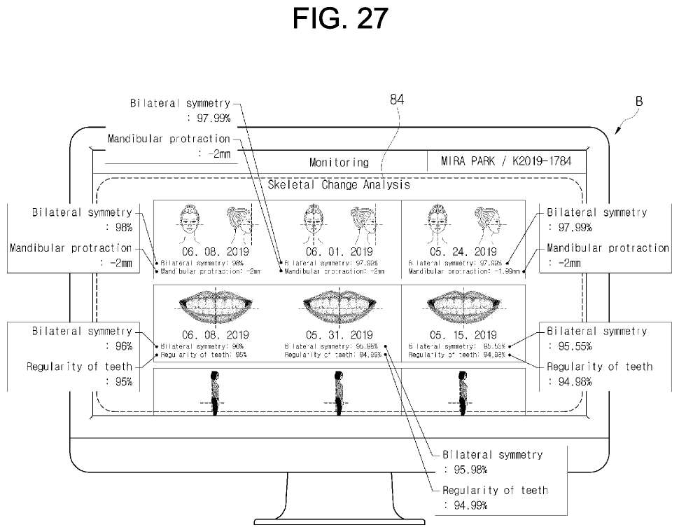

Finally, in FIG. 27, skeleton condition information about the selected user may be provided to the user skeletal state management page 84 which is to be turned to when an area regarding the skeletal status (Skeletal Change Analysis) is selected in the physical condition inquiry selection area 812.

When the above-mentioned administrator pages are used, there are advantages in that the administrator may continuously monitor the condition of the user and suggest the corrective direction to the user and vast big data related to the physical condition information about the user may be used for medical research.

The present disclosure has an effect of monitoring the imbalance state in the body, and more particularly, has an effect of measuring the state of the distribution of body muscle strength using a tape having a simple structure and providing data therefor.

In addition, the present disclosure has an effect of allowing a user to check a magnitude and direction of a force acting on the shape change of the entire human body structure by identifying, when a specific muscle generates a force of a specific direction and magnitude, an interrelationship with other muscles.

In addition, the present disclosure has an effect of providing a user with a risk of musculoskeletal disease by identifying a site where pressure is concentrated in a specific posture.

In addition, the present disclosure has an effect of correcting the imbalance of the body, and at the same time, allowing the user to check the process of correcting the imbalance.

In addition, the present disclosure has an effect of monitoring the sleep state.

Effects as not described herein may be derived from the above configurations. The relationship between the above-described components may allow a new effect not seen in the conventional approach to be derived.

In addition, exemplary embodiments shown in the drawings may be modified and implemented in other forms. The modifications should be regarded as falling within a scope of the present disclosure when the modifications are carried out so as to include a component claimed in the claims or within a scope of an equivalent thereto.

1. A method of providing a physical condition monitoring service using a physical condition measurement tape comprising a tape body selectively attached to a body and a physical condition measurement sensor provided on the tape body, a device, and a server communicating with the device, the method comprising:
performing first data transmission by wirelessly transmitting physical condition data from the physical condition measurement tape to the device;
performing second data transmission by transmitting the physical condition data from the device to the server;
performing physical condition data processing by analyzing and processing, in the server, the physical condition data into physical condition information; and
performing physical condition information transmission by transmitting the physical condition information from the server to the device.

2. The method of claim 1, wherein the physical condition measurement sensor comprises a muscle sensor configured to measure a tensile force formed on a body part of a user to which the tape body is attached, and
the physical condition information includes an average tensile force during a measurement time.

3. The method of claim 2, wherein the muscle sensor is provided on the tape body so that a longitudinal direction thereof intersects upper and lower lips of the user sequentially to detect mouth opening of the user, and
the physical condition information includes a time or number of times of mouth opening.

4. The method of claim 1, wherein the physical condition measurement sensor comprises a muscle sensor configured to measure a torsional stress formed on a body part of a user to which the tape body is attached, and
the physical condition information includes an average torsional stress during a measurement time.

5. The method of claim 1, wherein the physical condition measurement sensor comprises a breathing sensor configured to measure a breathing cycle during sleep of a user, and
the physical condition information includes a time or number of times of sleep apnea.

6. The method of claim 1, wherein the physical condition measurement sensor comprises a microphone configured to measure snoring that occurs during sleep of a user, and
the physical condition information includes at least one of a time, a size, and the number of times of snoring.

7. The method of claim 2, wherein the server further comprises a storage part configured to store the physical condition data and the physical condition information that are accumulated, and
the accumulated physical condition information is provided through the device.

8. The method of claim 7, wherein the server further comprises a statistics management part configured to process the accumulated physical condition data, and
the statistics management part processes the physical condition data into the physical condition information through extraction, calculation, and analysis of valid data.

9. The method of claim 1, further comprising:
performing photograph transmission by transmitting a body skeleton photograph of a user from the device to the server;
performing skeletal data processing by analyzing the body skeleton photograph and deriving skeletal information numerically indicating a state of skeletal balance, in the server; and
performing skeleton information transmission by transmitting skeleton information from the server to the device.

10. A physical condition measurement tape comprising:
a tape body selectively attached to a skin;
a physical condition measurement sensor provided on the tape body; and
a communication part provided on the tape body and configured to wirelessly transmit a value measured by the physical condition measurement sensor to an external device.

11. The method of claim 3, wherein the server further comprises a storage part configured to store the physical condition data and the physical condition information that are accumulated, and
the accumulated physical condition information is provided through the device.

12. The method of claim 4, wherein the server further comprises a storage part configured to store the physical condition data and the physical condition information that are accumulated, and
the accumulated physical condition information is provided through the device.

13. The method of claim 5, wherein the server further comprises a storage part configured to store the physical condition data and the physical condition information that are accumulated, and
the accumulated physical condition information is provided through the device.

14. The method of claim 6, wherein the server further comprises a storage part configured to store the physical condition data and the physical condition information that are accumulated, and
the accumulated physical condition information is provided through the device.
